(19) United States
(12) Patent Application Publication
(10) Pub. No.: US 2019/0183412 A1
(43) Pub. Date: Jun. 20, 2019

(54) SYSTEM AND METHOD FOR ASSISTING EXERCISING OF A SUBJECT

(71) Applicant: KONINKLIJKE PHILIPS N.V., EINDHOVEN (NL)

(72) Inventors: Laurentia Johanna Huijbregts, Eindhoven (NL); Jozef Hubertus Gelissen, Herten (NL); Hendrikus Petrus Maria Sterken, Deurne (NL)

(21) Appl. No.: 16/322,030

(22) PCT Filed: Aug. 8, 2017
(86) PCT No.: PCT/EP2017/070020

§ 371 Date: Jan. 30, 2019

(30) Foreign Application Priority Data
Aug. 8, 2016 (EP) 16183158.1

(51) Int. Cl. A61B 5/00; A61B 5/11; A61B 5/0205; A61B 5/0488; A61B 5/16

(52) U.S. Cl. CPC A61B 5/486; A61B 5/1118; A61B 5/0205; A61B 5/0488; A61B 5/02405; A61B 5/1122; A61B 5/1116; A61B 5/6817; A61B 5/165; A61B 5/112

(57) ABSTRACT

A system 1, a corresponding method and computer program for assisting exercising of a subject 7 is provided. The system comprises: an exercise state providing unit 10 for providing an exercise state of the subject 7 for or during an exercise session, a fatigue level determination unit 20 for determining a fatigue level of the subject 7 based on the exercise state of the subject 7, a fatigue level threshold determination unit 30 for determining a fatigue level threshold for the subject 7 for the exercise session, and an evaluation unit 40 for evaluating the fatigue level in comparison to the fatigue level threshold. It provides a more versatile system 1 for assisting exercising of a subject 7 and further provides an improved exercising assistance for the subject 7.

### FIELD OF THE INVENTION

The present invention relates to the field of assisting exercising of a subject. In particular, it relates to a system, a method and a computer program for assisting exercising of a subject. It finds application in improving sports performance, in particular in the field of running. However, it is to be understood that the present invention also finds applications in other fields and it is not necessarily limited to the above-mentioned application.

### BACKGROUND OF THE INVENTION

Although exercise is usually seen as healthy, reaching too far in exercising may be harmful to a subject, for instance, it can lead to muscle pain and longer recovery.

From WO 2014/135187 A1 a method, apparatus and computer program are known for estimating physiological states of subjects from gait measurements carried out during a physical exercise. The physiological state is completed from at least one of step interval variability and stride interval variability acquired from the gait measurements.

US 2010/0137748 A1 discloses a body motion detection section for continuously detecting the frequency of a user's activity as an activity level. The activity level detected by the body motion detection section is outputted to a fatigue detection section for estimating a user's fatigue level on the basis of the activity level.

US 2016/0030809 A1 discloses systems and methods for identifying and presenting information regarding a fitness cycle using earphones with biometric sensors. Fatigue level associated with fatigue experienced in response to a stimulus and recovery from such fatigue may be determined based on heart rate variability (HRV) data and learned user characteristics. One or more cycles of fatigue and recovery can be identified as a fitness cycle(s), each fitness cycle encompassing a period of time beginning with the stimulus associated with the fitness-related activity and progressing through recovery from the fatigue experienced in response to the stimulus associated with the fitness-related activity. Information regarding the fitness cycle(s) can be presented to a user in a variety of ways, including on a display of a computing device in communication with the earphones with biometric sensors.

WO 2015/069124 A1 discloses an exercise coaching system and a method of monitoring an activity session comprising receiving activity data indicative of at least one activity performed during the activity session, the activity data comprising a plurality of measurements associated to a plurality of parameters monitored during the activity session; a processor comparing at least some of the received measurements associated to at least two of the parameters with at least one set of a plurality of sets of measurements stored on a tangible computer readable medium; and a processor generating a training plan based at least partly on a comparison between the received measurements and the stored measurements.

GB 2 415 788 A discloses determining exercise level or fatigue by measuring electromyographic (EMG) signals produced by active muscles, measuring other parameters related to exercise and calculating at least one index indicative of exercise from the measured values (e.g. economy index, fatigue index). The other parameters may include electrocardiography (ECG) measurements, body movements (measured by inertia sensors such as accelerometers), and external conditions such as weather, altitude and terrain. The index allows exercise at different times or in different circumstances to be compared. Parameter sensors may be incorporated into an item of clothing (e.g. shorts). Feedback on exercise performance is provided to the user via a display.

However, despite estimating the physiological state of the subject, the state of the art does not assist the subject in exercising. Yet, the desire exists to relate physiological state to exercise efficiency.

### SUMMARY OF THE INVENTION

It is thus an object of the present invention to provide a system for assisting exercising of a subject which is more versatile. It is a further object of the present invention to provide an improved exercising assistance for the subject.

In a first aspect of the invention a system for assisting exercising of a subject is provided. The system comprises: a) an exercise state providing unit for providing an exercise state of the subject for or during an exercise session, b) a fatigue level determination unit for determining a fatigue level of the subject based on the exercise state of the subject, c) a fatigue level threshold determination unit for determining a fatigue level threshold for the subject for the exercise session, and d) an evaluation unit for evaluating the fatigue level in comparison to the fatigue level threshold.

Since the evaluation unit evaluates the fatigue level of the subject in comparison to a fatigue level threshold, wherein the fatigue level threshold is determined for an exercise session, the exercising of the subject can efficiently be assisted. In particular, since an evaluation result based on the fatigue level of the subject, which itself is based on the exercise state of the subject, is available, the exercise state of the subject can be evaluated versus a threshold for the exercise session and for the particular subject. More explicitly, the fatigue level threshold is a variable threshold and no fixed or predetermined value, but determined for a specific exercise session for the specific user. In other words, the exercise state of the subject, which can for instance be indicative for an exercising result or outcome, is employed in determining a fatigue level which is then evaluated with respect to a fatigue level threshold for determining an evaluation result. Accordingly, the exercising result or outcome can preferentially be assisted in an improved way.

An exercise session refers to a point in time or to a period of time, for or during which the user performs, performed or will perform exercise of any kind, for instance sports. The exercise session corresponds to a time point or time period, for which the fatigue level and/or the fatigue level threshold is determined. The determination can take place in real time, i.e. at the beginning or during the exercise session, in the retrospective, i.e. for a previous exercise session, and in the future, e.g. the fatigue level threshold can be determined for a future exercise session. The concept of the invention is not limited to the concept of exercise sessions and can be extended to a determination for an arbitrary point in time, referred to as a time of determination, which has, however, to be the same for the determined fatigue level and fatigue level threshold in order to be evaluated in a beneficial way.

The exercise state providing unit can be a storing unit, in which the exercise state of the subject is stored already, wherein the exercise state providing unit can be adapted to provide the stored exercise state of the subject. In this embodiment, the time of determination of the exercise session refers to a previous time, during which the exercise state stored in the exercise state storing unit has been provided. However, the exercise state providing unit can also be a receiving unit for receiving an exercise state of the subject from an exercise state measuring unit. In this example, the exercise session can substantially be real-time during a workout or exercising of the subject.

Preferentially, the exercise session for which the fatigue level threshold determination unit determines the fatigue level threshold for the subject corresponds to the exercise session, for which the exercise state providing unit provides the exercise state of the subject. Thereby, the evaluation by the evaluation unit can be based on corresponding exercise sessions, i.e. for corresponding times of determination.

The exercise state providing unit, the fatigue level determination unit, the fatigue level threshold determination unit and the evaluation unit can in one embodiment be provided in one or more processors that are arranged in the same or different physical devices. More precisely, the exercise state providing unit, the fatigue level determination unit, the fatigue level threshold determination unit and the evaluation unit can in one embodiment be provided together in a single device or in a different embodiment be distributed over multiple devices.

In one embodiment the fatigue level determination unit, the fatigue level threshold determination unit and the evaluation unit are adapted for communicating with the exercise state providing unit in a wired or wireless manner as well known in the art. In one embodiment, one, more or all of the exercise state providing unit, the fatigue level determination unit, the fatigue level threshold determination unit and the evaluation unit are provided at a server which is arranged for communicating with the rest of the system for assisting exercising of a subject by suitable communication means, for instance via the Internet.

Preferentially, a fatigue level of the subject indicates how far the exercise state of the subject at a time of determination for or during the exercise session is from the exercise state of the subject at a time of full recovery. Further preferentially, the fatigue level can be used for assessing a training effect of the subject. In principle, while the fatigue level can in one example be regarded an indication for efficient training, reaching too far, i.e. arriving at an excessive fatigue level, may be harmful to the subject, possibly result in muscle pain and long recovery needed. In other examples, a low fatigue level can indicate a less than desired exhaustion, which will also not yield an efficient training effect. For instance, the effect of overreaching during exercise can, according to the invention, advantageously be addressed through evaluating the fatigue level with a fatigue level threshold. Preferentially, the fatigue level threshold indicates in this example a fatigue level which should be achieved or not exceeded by the subject during exercising, wherein also other examples for fatigue level thresholds are contemplated.

Further preferentially, the fatigue level threshold indicates a preferred fatigue level which carries the most efficient training effect for the subject. Since the evaluation unit evaluates the fatigue level in comparison to the fatigue level threshold, and since the fatigue level threshold preferentially indicates a relevant condition during exercising, the exercising of the subject can be assisted in an improved way.

Preferentially, the evaluation result indicates a relation between fatigue level and fatigue level threshold, such as whether the fatigue level crosses the fatigue level threshold, a relative and/or absolute distance between fatigue level and fatigue level threshold and so on.

In one embodiment the fatigue level threshold determination unit is adapted to determine the fatigue level threshold based on at least one of an exercise history of the subject, a planned activity and a parameter of the subject.

Since the fatigue level threshold determination unit determines the fatigue level threshold based on at least one of an exercise history of the subject, a planned activity and a parameter of the subject, the fatigue level threshold is no generic threshold but takes into account the specific circumstances of the subject. The exercise history of the subject can indicate how much he/she exercised or trained in the previous days. For example, if the subject experienced high intensity exercises in the recent past, he/she will still not be fully recovered and thus will be more likely to overreach during the subsequent exercise. In this case, the fatigue level threshold determination unit preferably determines the fatigue level threshold to be lower than in case the subject is fully recovered. A planned activity can comprise, for instance, a high intensity exercise such as including a race in the near future. In this case, since the subject preferentially faces the future high intensity exercise fully recovered, the fatigue level threshold for the exercise session under consideration can, for instance, be lowered such that the time of recovery from the exercise at the exercise session under consideration will become shorter. A parameter of the subject can comprise an illness condition of the subject, drugs to be taken by the subject, et cetera. For example, the illness condition can include whether the subject was ill yesterday or in the recent past, he/she did sleep well or not, has gained weight during the past days, has muscle pain, etc. However, this list is not exclusive and in other embodiments the fatigue level threshold determination unit can be adapted to determine the fatigue level threshold based on alternative or additional parameters.

In one embodiment the determination based on the planned activity includes at least one of periodization and tapering prior to a future activity.

Tapering is the practice of reducing exercise in the days just before a high profile exercise, such as an important competition. The underlying principle is that in the period of tapering the body of the subject recovers to release optimal performance in the future activity, such as the important competition, for instance a race. Periodization refers to blocks in time of low, moderate and high exercise intensity, with the goal to get in optimal condition, usually for a planned future activity. The blocks in time used for periodization usually last for several weeks. Preferentially, since the fatigue level threshold determination unit considers periodization and tapering prior to future activity, the fatigue level threshold can be adapted for the exercise session under consideration such that a performance of the subject can be optimized at a future time of determination, i.e. a future exercise session. This future time of determination preferentially corresponds to the time of the future activity.

In one embodiment the fatigue level threshold determination unit is adapted to determine a maximum fatigue level threshold and/or a minimum fatigue level threshold.

Since the fatigue level threshold determination unit preferentially determines a maximum fatigue level threshold, this maximum fatigue level threshold can be taken as an upper limit for the fatigue level of the subject during exercising. Accordingly, this maximum fatigue level threshold can be taken as an indicator of overreaching or overtraining for the subject for or during the exercise session. Further, since the fatigue level threshold determination unit preferentially determines a minimum fatigue level threshold, this minimum fatigue level threshold can be taken as an indicator below which the subject does not or hardly experience a training effect. In other words, until the fatigue level of the subject for or during the exercise session does not exceed the minimum fatigue level threshold, no or hardly any training effect will be noticeable.

In one embodiment the fatigue level threshold can be set and/or influenced by a user. Preferentially, the subject itself or a different user, such as a coach or medical advisor, can set, i.e. arbitrarily define, or influence, i.e. increase or decrease, the fatigue level threshold which is provided by the fatigue level threshold determination unit. The fatigue level threshold is, in this embodiment, based on the setting and/or influence by the user.

In one embodiment the exercise state providing unit is adapted to provide the exercise state of the subject based on at least two parameters, wherein the at least two parameters correspond to at least two different of the following groups: i) speed, heart rate and heart rate variability, ii) running dynamics, iii) foot landing, iv) posture, and v) electromyography (EMG) related parameters.

Since the at least two parameters correspond to at least two different groups, the accuracy of the provided exercises state can be increased. Since furthermore the fatigue level determination unit determines the fatigue level based on a more accurate exercise state of the subject, also the fatigue level determination becomes preferably more accurate. In further preferred embodiments, the exercise state of the subject comprises parameters corresponding to more than two of the groups indicated above.

An EMG measures the electrical activity produced by skeletal muscles. The electrical activity produced by skeletal muscles is related to, for instance, muscle tension and thus also provides a parameter useful for being related to the exercise state of the subject.

In the following description, parameters based on a running activity of the subject are described. However, it is to be noted that of course other activities apart from running are contemplated for the system according to the invention.

Speed parameters can, for instance, be determined by means of a positioning sensor, such as a GPS system. However, in other embodiments, speed parameters can also be deduced from dynamic parameters, such as data collected by an accelerometer and/or a gyroscope.

It is known to determine heart rate from electrical or optical heart rate measurements of the subject, for instance. Heart rate variability (HRV) is derived from so called inter beat intervals between every heart beat and the next. For determining HRV, preferentially a chest belt like sensor using the electrical signal of the heart is employed. Algorithms, which have been discussed in the art, make use of speed of the subject, duration of the exercise, heart rate and heart rate variability only. Examples of such algorithms are, for instance, provided in publications by Firstbeat Technologies Ltd. titled “EPOC based training effect assessment” (Published: May 2005 and available via https://www.firstbeat.com/app/uploads/2015/10/white_paper_training_effect.pdf) and “Indirect EPOC prediction method based on heart rate measurement” (Published: May 2005 and available via https://www.firstbeat.com/app/uploads/2015/10/whitepaper_epoc.pdf).

Relying on speed, duration, heart rate and HRV for providing the exercise state only shows to be difficult in some situations. HRV is not always available, for instance during motion, and in particular when optical heart rate sensors are used, heart rate and HRV are not always be reliably available. Further, heart rate and HRV can change due to other factors not related to the fatigue level of the subject. For example, if an unexpected or surprising event, such as the subject being scared up by a dog or wild animal, happens, heart rate will go up and HRV will go down, without the subject being more fatigued or tired. Accordingly, the exercise state providing unit advantageously provides at least two parameters corresponding to at least two groups, such that fatigue level determination can become more reliable.

In this embodiment the determination will also be successful, even if one underlying sensor, which provides one of the parameters of the exercise state, would fail.

Preferentially, one or more accelerometers can provide information on running dynamics, and/or posture. However, also different sensors can be used for obtaining parameters corresponding to the above indicated groups, such as, for instance, one or more gyroscopes.

In one embodiment accelerometer and pressure sensors in a shoe of the subject are provided to provide running dynamics parameters, in particular a cadence of the subject, and foot landing parameters, in particular the position where on the foot the landing takes place, to the exercise state determination unit. Running dynamics and foot landing do not only depend on pace, i.e. the speed of the subject, but also on how tired the subject is, i.e. on the fatigue level of the subject. In particular, a subject with a higher fatigue level will generally land more on his heel, compared to the same subject being at a low fatigue level. In one embodiment parameters corresponding to the group of running dynamics comprise a ground contact time, a vertical oscillation, a cadence, a stride length, a stride interval, a stride interval variability, a left-right balance, a measure for braking, a step length, a step interval and a step interval variability.

In this embodiment ground contact time is understood as a measure of the amount of time a foot of the subject stays on the ground during each step. Vertical oscillation is a measure, for instance by means of an accelerometer, which indicates motion of the subject in the vertical direction. Preferentially, cadence identifies a number in steps per minute, as how frequently the foot of the subject contacts the ground per minute. A step length is a distance from initial contact of one foot to the next initial contact of the opposite foot, wherein a stride length is the distance from initial contact of one foot to the next initial contact of the same foot. Stride/step interval refers to the distance in time between two consecutive strides/steps, stride interval variability and step interval variability refer to the variability of the stride interval and the step interval, respectively. Braking, in this embodiment, relates to the change in horizontal velocity, for instance derived from a horizontal accelerometer, and indicates a decrease in speed the subject experiences on each step. Left right balance can refer to any deviation from a symmetrical movement of the subject, for example a difference in step length, ground contact time, or braking; possible quantifications are the length of a step with the right foot minus the length of a step with the left foot, the ratio of the two, etc.

In one embodiment parameters corresponding to the group of posture comprise an angle of upper body, a pelvic rotation and a head orientation.

An angle of upper body can, for instance, be determined by means of a sensor attached to the upper body of the subject. Preferentially, pelvic rotations indicates an amount the subjects' pelvis moves on three axes, being a tilt axis, i.e. a forward/backward movement, a drop axis, i.e. up and down movement, and rotation, i.e. left and right movement. Preferentially, the head orientation includes an angle under which the head is bent or turned with respect to the neck and/or the body.

In one embodiment the exercise state providing unit is adapted to provide the exercise state of the subject based on at least one of a foot landing parameter and a head orientation parameter, wherein the exercise state providing unit is configured to determine the at least one of a foot landing parameter and a head orientation parameter based on an inertia signal.

An inertia signal preferentially relates to a change in the state of motion of an inertia sensor from which the inertia signal originates. Preferentially, the state of motion comprises velocity, direction and/or angular momentum. The inertia signal preferentially comprises at least one of an accelerometer signal and a gyroscope signal.

Preferentially, the foot landing of the subject can be determined with an accelerometer since landing on the heel can result in more impact and more braking than landing on the front foot. Further preferentially, the inertia signal such as the accelerometer signal or gyroscope signal originates from a position on the head of the subject, such as from a sensor mounted in-ear of the subject, such that the head orientation can be accurately determined.

In one embodiment the exercise state providing unit comprises an exercise state measurement unit. The exercise state measuring unit can preferably comprise one or more sensors for measuring one or more parameters of the exercise state of the subject.

In one embodiment the exercise state measurement unit comprises an in-ear sensor adapted to be mounted in the ear of the subject. Preferably, the exercise state measurement unit comprises an optical heart rate (OHR) sensor, wherein the OHR sensor is adapted to be mounted in the ear in the subject. Further preferably, the exercise state measurement unit comprises in this embodiment an accelerometer for measuring motion of the subject. The additional motion signal can advantageously be employed in improving the OHR signal and/or to derive additional parameters, such as at least one of a running dynamics parameter or even an orientation of the head of the subject. The orientation of the head, such as expressed as a head angle, can give an indication of fatigue, as some runners bend their neck backwards when they get tired, while others start to look more down when they get tired.

In one embodiment the exercise state measurement unit comprises an OHR sensor, which is comprised within a wrist-worn device, such as a watch. Preferably, in this embodiment, the exercise state measurement unit comprises a patch for being mounted to the upper leg of the subject. Preferably, the patch includes an accelerometer for measuring running dynamics of the subject and an EMG sensor to measure leg muscle fatigue.

In one embodiment the exercise state measurement unit comprises a GPS sensor, such as a GPS sensor comprised in a sports watch or a smartphone, wherein the GPS sensor is adapted to obtain the speed of the subject. In other embodiments, additionally or alternatively, the exercise state measurement unit comprises an accelerometer for determining the speed of the subject. Preferably, in case the exercise state measurement unit comprises a GPS sensor, the GPS sensor provides additional information on the location which can be employed in determining whether the subject is running or moving on a flat surface, uphill or downhill, or even additional environmental information, such as weather information, can be determined by exercise state providing unit in this embodiment. In one embodiment additional sensors on the sports watch or the smartphone, such as an air pressure sensor for measuring wind and altitude changes and a thermometer for measuring temperature, can be elements of the exercise state measurement unit and provide data relevant to the exercise state of the subject.

In one embodiment the exercise state measurement unit comprises a chest strap comprising an accelerometer to derive running dynamics, posture and speed, and electrodes to measure heart rate and heart rate variability (HRV). In this embodiment, data from the chest strap can preferably sent by wired or wireless means to the remaining units which are, for instance, implemented in a sports watch, smart glasses, or a smartphone of the subject.

In one embodiment the exercise state measurement unit comprises a clip containing an accelerometer for determining running dynamics and/or posture. Preferably, the clip is arranged for always being attached to the same location on the subject's body in order to allow for a good learning of running dynamics and posture. Preferably, the clip is adapted to be attached to the cloths, such as shorts, of the subject.

In one embodiment the exercise state measurement unit comprises one or more pressure sensors to measure the foot landing of the subject. Additionally or alternatively, the foot landing of the subject can be determined with an accelerometer since landing on the heel can result in more impact and more braking than landing on the front foot.

In one embodiment the fatigue level determination unit is adapted to provide the fatigue level of the subject based on past exercise states of the subject.

Past exercise states of the subject can, for instance, be stored on the system itself, or, in another embodiment, be stored on a remote computer, such as a server which is connected to the system via the Internet. Preferably, the fatigue level determination unit is adapted to receive the past exercise states of a subject from the server via suitable communication means. Preferentially, the fatigue level determination unit is adapted to process the past exercise states of the subject with machine learning algorithms, such that the fatigue level determination unit learns from past exercise states for the determination of the fatigue level. Instead of past exercise states originating from real exercising data of the subject, the fatigue level determination unit can further be adapted to base the determination on exercise states of the subject which are provided to the system in a manual form, such as inputted by the subject itself.

In one embodiment the fatigue level determination unit is adapted to provide a reference exercise state of the subject and to provide the fatigue level based on a deviation of the exercise state from the reference exercise state.

Since the fatigue level determination unit provides a reference exercise state of the subject, wherein the reference exercise state preferentially corresponds to an exercise state of the subject in a completely recovered state, a deviation of the exercise state correlates with the fatigue level of the subject, since in case the subject would be addressed, the exercise state would be equal to the reference exercise state. In one embodiment, the reference exercise state is derived from past exercise states of the subject, such as preferentially through machine learning. In other embodiments, the reference exercise state can be provided manually by the subject, for instance.

In one embodiment the exercise state and the reference exercise state of the subject each comprise a set of at least two parameters, wherein the fatigue level determination unit is adapted to provide the fatigue level based on a weighted sum of differences between each set of corresponding parameters among the reference exercise state parameter set and the exercise state parameter set.

In this embodiment all parameters preferably are numerical values or can be represented with numerical values. Advantageously, since a weighted sum of differences is employed for determining the fatigue level based on each out of the corresponding parameters, the relative influence of a respective parameter for the fatigue level of the subject can be accounted for. Preferably, a higher fatigue level indicates the subject being more fatigued or tired. Accordingly, the higher an influence of a respective parameter, the higher the corresponding weighting factor would be.

In some embodiments, in case the reference exercise state comprises multiple parameters, the exercise state lacks one or more of the parameters comprised in the reference exercise state. For instance, a sensor which is to measure the respective parameter with the subject is not in good contact with the subject or does not provide information for other reasons, such as a missing GPS connection for a GPS sensor for speed determination. In these embodiments, the respective one or more parameters which are lacking from the exercise state can be given a zero weighting factor. In this example, the overall fatigue level will be lower than when considering all parameters since the weighted sum consists of fewer terms. In an alternative embodiment, the weighting factors of the remaining parameters can be adapted to compensate for the lacking parameters, such that the fatigue level provided by the fatigue level determination unit based on the exercise state with missing parameters corresponds to the fatigue level provided by the fatigue level determination unit based on the exercise state with a complete set of parameters.

In one embodiment the fatigue level determination unit is adapted to provide a plurality of reference exercise states for the subject. For instance, the plurality of reference exercise states of the subject could all be based on different values for a particular parameter of the exercise state, for instance, the speed of the subject. In this embodiment, multiple reference exercise states for the subject could be provided for different velocities, such as 10 kilometers per hour, 11 kilometers per hour, et cetera. However, in other examples, also different parameters can be used for the different reference exercise states. Preferentially, to accommodate for intermediate parameters, interpolation between two or more of the reference exercise states can be carried out.

In one embodiment the weighting factors can be learnt through machine learning from past exercise states. In another embodiment, the weighting factors can be manually set by the subject or another user.

In one embodiment the fatigue level determination unit is adapted to determine the fatigue level based on environmental influences and/or the fatigue level threshold determination unit is adapted to determine the fatigue level threshold based on environmental influences.

Advantageously, since the fatigue level and/or the fatigue level threshold is determined based on environmental influences, the fatigue level and/or the fatigue level threshold can be determined more accurately, since influences not indicative of the fatigue level of the subject are not considered in the determination. Environmental influences comprise wind, inclination, altitude and temperature, and are, in one embodiment, derived from an environmental measuring unit such as sensors worn by the subject, or, in another embodiment, additionally or alternatively provided by an environmental parameter providing unit, wherein the environmental parameter providing unit provides environmental influence data from data in the cloud, when the location of the subject is known. Correlation between environmental factors and the exercise state of the subject, which can in one embodiment be known from experience from other subjects or derived from literature, for instance, or, in another embodiment, learnt from the subject's response to certain environmental parameters, is advantageously exploited. In the first alternative, the fatigue level determination unit exploits these correlations to either increase or decrease the fatigue level, depending on whether environmental influences are favorable, e.g. tail wind or running downhill, for instance, or unfavorable, e.g. head wind, soft ground, or running upwards. In the alternative, the fatigue level threshold determination unit can adapt the fatigue level threshold based on the recognized correlations between exercise state and environmental influences, for instance increase the fatigue level threshold in case the subject performs an uphill run, and the like. However, these adaptations to environmental influences are of course not limited, and other adaptations based on environmental influences are contemplated by the skilled person.

In one embodiment the system further comprises a user notification unit for notifying a user of the evaluation of the fatigue level.

Preferably, the user notification unit is adapted to notify the user of the result of evaluation. For instance, the user notification unit can notify the user of the evaluation result, in order for the user to evaluate the training effect of the subject. It should be noted that the user can be the subject, i.e. the person whose exercise state is considered, or a different person, such as a coach and/or a physician.

In case the system is intended to be used as an over-exercise indicator, for instance, the user notification unit can be adapted to notify the subject in case the evaluated fatigue level exceeds the fatigue level threshold. In this embodiment, the user can be a user monitoring the underlying exercise data at a later stage, or can be the subject itself while using the system such as the subject during exercising. Preferentially, in case the time of determination is substantially in real-time, i.e. the subject carries the system for assisting exercising along while performing the exercise, the user notification unit notifies the user upon reaching the fatigue level threshold such that the subject can stop exercising to achieve the most beneficial exercise effect, without suffering from long recovery or muscle pain, or the like. However, the user notification unit can, in other embodiments, also be adapted to notify the user at different evaluation results, such as a distance from fatigue level to fatigue level threshold or that the fatigue level threshold has not been reached yet, for instance.

In one embodiment the user notification unit comprises a display, wherein the notification is done visually. For instance, the display is provided with a wrist-worn watch device, integrated into smart glasses or implemented with the display of a portable mobile phone of the subject. A notification can, for instance, comprise a visual warning, such as a particular color, or can be absent dependent on the evaluation result. In other embodiments, also other notifications than visual notifications, such as acoustical, vibratory, or the like, are contemplated.

In one embodiment the system comprises a user notification unit for notifying the user of the evaluation of the fatigue level, wherein the user notification unit comprises an acoustical notification unit adapted to be mounted in-ear of the subject.

In this embodiment advantageously a combination of acoustical notification and sensors, such as for instance for determining parameters of the exercise state of the subject, can be implemented. In one embodiment, the acoustical notification unit is adapted to notify the user with a sound, wherein the information is provided to the user at a different position, such as visually on a screen, for instance, on the subject's watch or phone display or, in case of later analysis, a screen the user is looking at. However, in other embodiments, the acoustical notification unit itself can be adapted to provide the notification by voice, such as using a voice synthesizer.

In a further aspect a method for assisting exercising of a subject is provided. The method comprises: a) providing, by an exercise state providing unit, an exercise state of the subject for or during an exercise session, b) determining, by a fatigue level determination unit, a fatigue level of the subject based on the exercise state of the subject, c) determining, by a fatigue level threshold determination unit, a fatigue level threshold for the subject for the exercise session, and d) evaluating, by an evaluation unit, the fatigue level in comparison to the fatigue level threshold.

In a further aspect a computer program for assisting exercising of a subject is provided. The computer program comprising program code means for causing a system as defined in claim 1 to carry out the method as defined in claim 14, when the computer program is run on the system.

It shall be understood that the system of claim 1, the method of claim 14 and the computer program of claim 15 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system for assisting exercising of a subject,

FIG. 2 shows schematically and exemplarily an analysis of exercise states recorded over time, and

FIG. 3 shows a flowchart exemplarily illustrating an embodiment of a method for assisting exercising of a subject for the system for assisting exercising of a subject shown in FIG. 1.

### DETAILED DESCRIPTION OF EMBODIMENTS

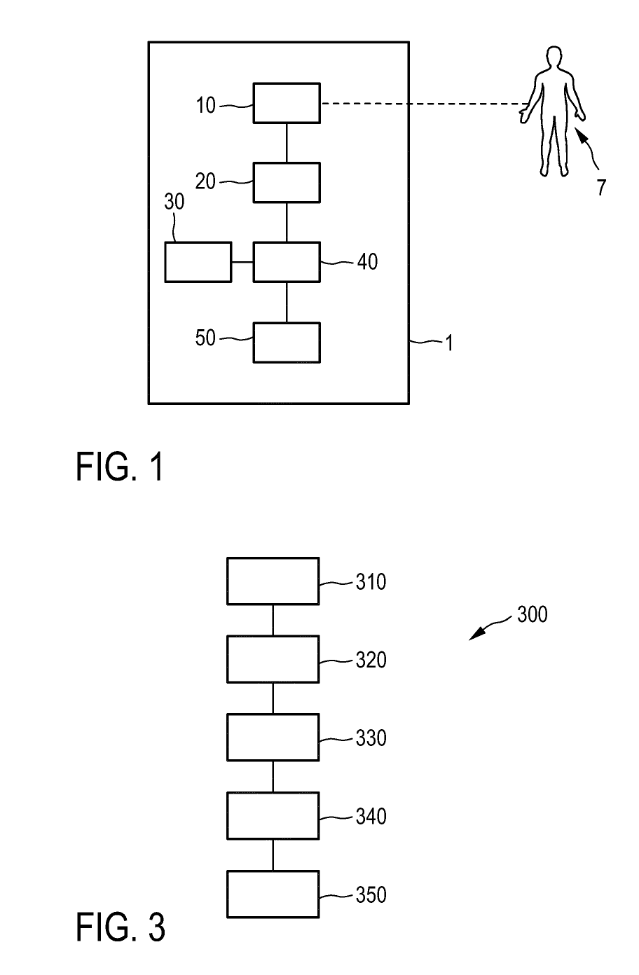

FIG. 1 shows schematically and exemplarily a system 1 for assisting exercising of a subject 7. System 1 comprises an exercise state providing unit 10 for providing an exercise state of subject 7 at a time of determination, a fatigue level determination unit 20 for determining a fatigue level of the subject based on the exercise state of subject 7, a fatigue level threshold determination unit 30 for determining a fatigue level threshold for the subject at the time of determination, an evaluation unit 40 for evaluating the fatigue level in comparison to the fatigue level threshold, and a user notification unit 50 for notifying a user of the evaluation of the fatigue level. The time of determination corresponds in the context of this patent application to an exercise session, which the subject exercised in the past, is currently exercising substantially in real time or plans to exercise in the future.

In this example, system 1 is comprised within one device which is preferably attached to, for instance, a wrist of subject 7. In this example, since system 1 can be attached to the wrist of subject 7, the user who is notified by user notification unit 50 is the subject 7 itself.

In this example, exercise state providing unit 10 is adapted to provide an exercise state of the subject substantially in real-time while the subject 7 is exercising. For this purpose, exercise state providing unit 10 is an exercise state measuring unit adapted to measure at least one exercise state relevant parameter of the subject 7. For instance, in this example exercise state providing unit 10 comprises an optical heart rate (OHR) sensor which is to be integrated in earbuds for playing music, or the like. The OHR can determine a heart rate of the subject based on optical measurements.

In this example, exercise state providing unit 10 further comprises at least one accelerometer which is attachable to at least one of the wrist, the earbud, a chest strap or a shoe of subject 7. Since the accelerometer is provided and in this example attached next to the OHR sensor, motion-induced noise can be filtered from the signal in order to obtain the correct heart rate of subject 7. This applies further to the alternative example in which an optical heart rate sensor is attached to the wrist instead of the earbud described in this example. At the same time, the accelerometer is adapted to give additional motion features of subject 7, like, for instance, the subject's 7 step frequency, called cadence in the context of running. In general, accelerometers can provide information on posture which is derived from the orientation of the accelerometer at the place where it is positioned, the kind of activity, e.g. distinguish running from cycling, et cetera, and, in the example of running, running dynamics such as ground contact time, vertical oscillation, cadence, stride length, and left-right balance, for instance. Depending on which parameter is preferred, the position of the accelerometer can be chosen to be one or the other. Even further, accelerometers can be employed in obtaining information on braking parameters, i.e. a change in horizontal velocity leading to a decrease in speed the subject 7 experiences for every step, and pelvic rotation parameters.

In other examples, exercise state providing unit 10 can provide additional or alternative sensors which include a gyroscope, a magnetometer and a barometer. This list is of course not limited to the examples given above, and alternative or additional sensors can be provided in other examples. For instance, pressure sensors can be provided in a shoe of subject 7 to determine where on the foot a landing takes place for each step. Further, in other examples, EMG sensors can be provided which measure the electrical activity produced by skeletal muscles. The electrical activity produced by skeletal muscles is related to muscle tension and muscle fatigue and thus also provides a parameter useful for determining an exercise state of subject 7.

Fatigue level determination unit 20 determines the fatigue level based on the exercise state. In particular, in this example, fatigue level determination unit 20 determines the fatigue level based on all the parameters defining the exercise state provided by exercise state providing unit 10. Different tendencies in different parameters comprised in the exercise state can indicate either a higher or lower level of fatigue. It is known that running dynamics parameters depend on the pace of subject 7; when a runner tries to run faster, his cadence and stride length will increase and ground contact time and vertical oscillation will decrease. Further, for instance, the foot landing can go more towards the front foot. However, the same subject 7 will, in case of a higher fatigue level, have a slower cadence, smaller stride length, larger ground contact time and will land more on the heel, compared to the same subject 7 in a completely recovered state. By incorporating multiple parameters comprised in the exercise state, fatigue level determination unit 20 determines the fatigue level more accurately than by just considering heart rate and/or pace taken by its own.

In further examples, also other circumstances, like environmental circumstances such as inclination, wind, temperature, et cetera, can be taken into consideration. Further, also subject 7 itself can influence some of the exercise state parameters, for instance increase the heart rate by actively talking and the like. Advantageously, all these circumstances can be considered and their influence on running parameters, foot landing, body posture, together with their relationship with heart rate and pace, can be assessed for determining the fatigue level more accurately.

Since fatigue level determination unit 20 determines the fatigue level based on multiple parameters of the exercise state provided by exercise state providing unit 10, compared to fatigue level determination units previously known in the art, the fatigue level can be reliably determined even if HRV is not available, for instance in case subject 7 does not wear a chest strap, or in case the heart rate (variability) or speed measurements are off, for instance, due to bad contact with the skin or a bad speed measurement connection, such as a bad GPS connection.

Fatigue level threshold determination unit 30 determines the fatigue level threshold for subject 7 for an exercise session set at the time of determination, in the example of FIG. 1, substantially for the current moment in time. The determination of the fatigue level threshold by fatigue level threshold determination unit 30 will be described in more detail further below.

The fatigue level threshold and the fatigue level of subject 7 are provided to evaluation unit 40 which evaluates the fatigue level in comparison to the fatigue level threshold. In this example, both the fatigue level and the fatigue level threshold are provided as numerical values which can easily be compared to each other. However, in other examples, the fatigue level threshold and the fatigue level can also be different measures, such that the evaluation can be more sophisticated.

In case evaluation unit 40 evaluates, for instance, a critical situation, such as the fatigue level exceeding the fatigue level threshold and thus indicating an over-exercise of subject 7, the result of evaluation is provided to user notification unit 50 and the user can be notified of the evaluation of the fatigue level. In this example, subject 7 can be provided a warning, for instance, to stop exercising. In one example, this warning can be performed with a sound, such as a spoken word, a beep, et cetera, or visually. In this example, subject 7 can be warned using the earbuds or a visual indication visible at the wrist-worn device or integrated in glasses of the subject. In this example, the notification provided by user notification unit 50 can be absent while the evaluation result indicates a non-conspicuous fatigue level of subject 7. Further, not only critical fatigue levels can be notified by user notification unit 50, for instance, in case the fatigue level approaches the fatigue level threshold, user notification unit 50 can notify subject 7 with different notifications, such as a different acoustic or visual notification. In case the fatigue level is close to the fatigue level threshold, i.e. the state where subject 7 should stop his or her exercise in this example, a sign with a different color can be provided on, for instance, the wrist-worn device. However, as mentioned before, also other forms of notification based on the evaluation result are contemplated.

The following particular example describes system 1 for assisting exercising of subject 7 in more details with the particular example of an over-exercise indicator. More particularly, while the following exemplary description particularly addresses running, in other examples, it could also be used for other sports or other applications, such as medical recovery applications which do not include sports.

In this example, exercise state providing unit 10 can gather data for heart rate, speed and duration of subject 7, for example from a) a sports watch containing an OHR sensor, and an accelerometer and/or GPS sensor. Accelerometer and/or GPS can be obtained from a sports watch connected to a chest strap (for heart rate and optionally HRV) or from a heart rate sensor connected to a phone using GPS. Accordingly, system 1 and particularly exercise state providing unit 10 can in these examples either be comprised in the sports watch or the phone and/or implemented by separate units or as computer code means.

The following description does not rely on HRV. This can be the particular case while an OHR sensor is used instead of a chest strap. However, the following example can likewise be implemented with the additional information of HRV.

From heart rate and speed data, the correlation between heart rate and speed for the subject in a fully recovered state, e.g. a fit condition not near the end of a training or race event, in which the subject 7 might already be tired, is learnt. These learnt conditions are employed by fatigue level determination unit 20 for determining the fatigue level based on the learnt correlations. For example, a learnt correlation for a particular subject 7 can be that, under normal environmental circumstances, the distance covered per heart beat is always close to 1.9 meters, wherein this holds for intervals between 800 meters and 10 kilometers, for instance. In other words, subject 7 can fairly easily, namely with a heart rate of 140 beats per minute, run 1 kilometer in 3 minutes and 46 seconds. The same subject 7 would run 1 kilometer in a race at a heart rate of 190 beats per minute and need only 2 minutes and 46 seconds. Preferentially, fatigue level determination unit 20 is adapted to vary the learnt correlation over time, i.e. on the time scale of weeks, for instance, when subject 7 is getting in shape or gaining weight. Considerable deviation from the learnt correlation is one indication of a higher fatigue level, i.e. an indication of reaching an over-exercise state. For the above example, if the same subject 7 would run 1 kilometer interval at a heart rate of 180 beats per minute and take 3 minutes and 15 seconds instead of the predicted 2 minutes and 55 seconds, subject 7 is probably at a high fatigue level. However, a fatigue level determination based solely on the correlation between heart rate and speed is not always very accurate, since external factors can influence the heart rate as well. For instance, in case subject 7 was talking enthusiastically to a running mate, frightened by a dog or the like, his heart rate increases, while the fatigue level of subject 7 could still be very low, such that fatigue level determination unit 20 should not determine a high fatigue level. For this reason, fatigue level determination unit 20 considers this correlation only with a certain weighting factor, for instance, as part of the algorithm to determine over exercising. Preferably, in case the heart rate sensor gives no or invalid data due to bad skin contact or low blood perfusion, the weighting factor used by fatigue level determination unit 20 for the heart rate versus speed relationship correlation used in the over-exercise indicator system should reduce to zero.

In this example, talking of subject 7 can be determined by accelerometers in earbuds or a microphone, and a corresponding parameter can be provided as part of the exercise state of the subject by exercise state providing unit 10. However, talking and the dog frightening are just examples for elevated heart rates which might have many causes, and the achievement of system 1 for assisting exercising of a subject 7 according to the present invention aims at coping with any of the causes. For this reason, exercise state providing unit 10 incorporates additional parameters for providing the exercise state of the subject, for instance, skeletal muscle output such as one or more of running dynamics, posture, foot landing and electrical muscle signals.

As already detailed above, running dynamics, posture, foot landing and electrical muscle signals can be determined by exercise state providing unit 10 using or comprising any of the sensors discussed above. However, also additional sensors, such as cameras, can be used for analyzing running dynamics, posture and foot landing. For instance, several cameras can be placed around a 400 meter running track in one example.

Most subjects 7, experiencing an elevated fatigue level, will show a decline in cadence, a decrease in step length, an increase in ground contact time, a sag in the hip, an increased inclination of the upper body forward, more pronounced differences between left and right and a lower medium frequency and higher amplitude of the EMG signal, and the foot landing will be more towards the heel, while a certain heart rate remains constant. In this example, one, more or all of these characteristics or correlations are integrated in the determination of the fatigue level by fatigue level determination unit 20. Fatigue level determination unit 20 is preferably adapted to, in addition to using the general relationships between skeletal muscle output and fatigue level, to learn subject-specific relationships. For example, if subject 7 for quite a number of trainings or exercises near the end gets a relatively high heart rate compared to his speed and at the same time experiences a change in upper body inclination from 0 degrees, i.e. completely vertical, to 10 degrees, i.e. bent forward, a body inclination around 10 degrees could be used as an important aspect, i.e. could be assigned with a high weighting factor, in the fatigue level determination. Additionally or alternatively to this automatic learning, fatigue level determination unit 20 can also be adapted to obtain an indication of experienced fatigue after each training for the learning process which can be inputted by the subject.

With different subjects 7, cadence might get more irregular when they get tired. Also this correlation could be learnt by fatigue level determination unit 20. Thereby, in case fatigue level determination unit 20 has determined such individual correlation, i.e. that for the specific subject 7 the cadence becomes more irregular, when he/she is getting tired, this could become one of the input parameters to the fatigue level determination.

Environmental factors like wind, inclination, altitude and temperature are in one example also considered by fatigue level determination unit 20. Wind, inclination, altitude and temperature can be derived from additional sensors worn by subject 7 or could be derived from data received by exercise state providing unit 10 or fatigue level determination unit 20 or a dedicated unit of system 1, for instance, from a remote server, such as the cloud, which are transmitted to system 1 via wired or wireless connections, such as WiFi or mobile data connections. Preferably, data in the cloud are indicative of weather and geography, when the location of subject 7 is known.

General relationships, i.e. averages over many subjects which are, for instance, derived from other users of system 1 or derived from literature, between environmental conditions and other parameters of the exercise state of subject 7, such as running speed reduction/increase, can be considered, while a deviation from these known relationships, for instance wind from the back without an increase of distance covered per heart beat, might indicate an increased fatigue level of subject 7 and thus give an indication of a state of over exercising. Alternatively or additionally, fatigue level determination unit 20 can learn the subject's 7 response to certain environmental conditions when the subject is fit versus when the subject is tired.

As indicated above, environmental factors like wind and inclination do not only influence the causal relationship between speed and heart rate, but, for instance, running uphill can change the foot landing more towards the front foot, decrease the stride length, increase their ground contact time, and bend the upper body more forward as compared to running on a flat surface for many subjects 7. Fatigue level determination unit 20 is adapted to distinguish these changes from similar changes due to an increased fatigue level. Therefore, fatigue level determination unit 20 is adapted to detect when subject 7 is running uphill, for instance by using an air pressure sensor which can measure a rise in altitude, or by using the location information from GPS or the mobile phone network and combining this with geographical information from the area. Fatigue level determination unit 20 can learn the subject's 7 usual running dynamics, foot landing, posture and heart rate versus speed correlation, for instance, for uphill running and, likewise, for downhill running. Deviations from this usual and learnt behavior are preferably used as an indication of an over-exercise state, i.e. lead to a higher determined fatigue level of subject 7. In one example, the subject-specific uphill running correlation can be determined by fatigue level determination unit 20 even as a function of the slope of inclination.

In cases subject 7 only sporadically runs uphill or for other reasons, such as subject 7 has recently started using system 1 according to the invention, a lack of learnt data to compare with when subject 7 is running uphill might occur, wherein subject 7 can be compared with uphill running data of similar subjects, i.e. subjects with similar running behavior on a flat surface as subject 7 currently using system 1, even more preferably these similar subjects also have comparable weight. Likewise, this holds mutatis mutandis also for different environmental parameters, such as running downhill or running with strong head or tail wind. Further, also the surface of the road subject 7 is running onto might have an influence on the fatigue level of the subject, since, for instance, running on sand or very soft ground might produce deviations from the usual running dynamics, even if subject 7 is not tired yet. Data indicating the surface conditions of a particular road or track can, for instance, be connected with map data received from the cloud, or the like. Also in this respect, data of similar subjects can be taken from the cloud, or the data might comprise known relationships from literature which are programmed into fatigue level determination unit 20.

Having described many factors which influence the fatigue level determination by fatigue level determination unit 20, it will now be described in further detail how this determined fatigue level is evaluated for assisting subject 7 in exercising. In this respect, first the determination of a fatigue level threshold by fatigue level threshold determination unit 30 is described in more detail. In general, the idea behind a fatigue level threshold for subject 7 is that in several cases it is beneficial to not always have a hard workout which requires a long recovery period, but instead the fatigue level reached in training should be varied from training to training. A fatigue level threshold for the subject which is determined by fatigue level threshold determination unit 30 can be based on input from or uploaded from the internet for each training. For instance, for each training a maximum or minimum allowed fatigue level could be provided. For example, general training schedules for guiding recreational runners towards their first marathon exist. These give indications of the distances that should be run on a weekly or daily basis. One can imagine that in the future such schedules could also contain fatigue thresholds.

As briefly stated above, user notification unit 50 can be adapted for notifying a user or subject 7, as in this example, in case the maximum allowed fatigue level, i.e. a maximum fatigue level threshold, is exceeded by the subject. This evaluation is performed by evaluation unit 40. It should be stated that alternatively or additionally, fatigue level threshold determination unit 30 can also provide a minimum fatigue level threshold, wherein the subject 7 or any user gets notified by user notification unit 50 in case subject 7 is far from a maximum allowed fatigue level or still below a minimum allowed fatigue level. In this example, user notification unit 50 can encourage subject 7 to go faster, for instance.

In particular, fatigue level threshold determination unit 30 can determine a different fatigue level threshold for different times of determination, i.e. different workouts or training sessions of subject 7.

In addition to being user-defined or based on user input, the fatigue level threshold determined by fatigue level threshold determination unit 30 can, for instance, depend on training load in the past couple of days, a scheduled training load for the upcoming couple of days, an upcoming race, periodization and/or personal circumstances like illness. In other words, a fatigue level threshold which is determined by fatigue level threshold determination unit 30 depends on a set goal and/or exertion that has taken place during the last hours or days for subject 7. Examples of goals are, as indicated above, race, normal training, easy training, or the like. Subject 7 would select easy training if he/she has done or planned intensive trainings or races in the preceding or following days or even on the same day. In this example, fatigue level threshold would be rather low and evaluation unit 40 would, for instance, rather early indicate the subject as approaching a status of over-exercising. In the contrast, subject 7 could also select race, wherein fatigue level threshold determination unit 30 would determine a very high fatigue level threshold such that user notification unit 50 does not issue any warnings or the like until subject 7 approaches a fatigue level which is getting dangerous to his/her health.

Additionally or alternatively to the user indicating the maximum allowed fatigue level threshold for the upcoming training, fatigue level threshold determination unit 30 can determine the fatigue level threshold automatically based on, for instance, exertion during the preceding days or about to expect from the upcoming days from its stored data and/or from data received via the cloud, et cetera. For example, in case fatigue level threshold determination unit 30 determines a very intensive training the day before, the fatigue level threshold would be set to a comparably low value, such that evaluation unit 40 would evaluate subject 7 to be in an over-exercise state already with a comparably low fatigue level. Even further, the fatigue level threshold can be determined based on complete planned periodization which is given by manual input of the subject. This input can, for instance, be entered on a distant computer and system 1 could acquire this input from the cloud or any other transmission means. With this input, in case subject 7 is in a high training load time block, the fatigue level threshold will be determined higher by fatigue level threshold determination unit 30 than during a light training load time block.

Next, an example determination of the fatigue level by fatigue level determination unit 20 will be described in further detail. In this example, fatigue level determination unit 20 implements a formula which calculates the fatigue level based on two or more of the above-mentioned parameters of the exercise state of the subject provided by exercise state providing unit 10. The output of the formula is compared to the fatigue level threshold by evaluation unit 40 and, depending on the evaluation result, the user is notified or not by user notification unit 50. In the following example, a fatigue level threshold corresponding to a high fatigue level allowed could equal to a numerical value of 50, while a threshold could equal the numerical value of 10, when only a very low fatigue level would be allowed. However, in other implementations or examples, these numerical values can of course be different. Further, in other examples, also different classification schemes apart from numerical values can be implemented.

In this example, the formula considers learnt values for several parameters for a particular subject 7, when subject 7 is rested, i.e. not yet tired, wherein the subscript “r” will be used for these learnt parameter values addressed. The number of different parameters that is comprised in the exercise state of the subject will be referred to as N. In the following table of learnt data for a rested (i.e. not yet tired) subject 7, nine parameters, namely speed (sp), heart rate (HR), cadence (ca), left-right balance (lrb), foot landing (fl), upper body angle (uba), pelvic rotation (pr), EMG frequency (Ef) and EMG amplitude (Ea) are provided, such that N=9.

| | | | | | upper | | | |
|---|---|---|---|---|---|---|---|---|
| | heart | | left- | | body | pelvic | EMG | EMG |
| speed | rate | cadence | right | foot | angle | rotation | frequency | amplitude |
| (km/h) | (bpm) | (bmp) | balance | landing | (°) | (°) | (Hz) | (V) |
| 10 | 115 | 160 | 1.0 | heel | 5 | 20 | 160 | 1.1 |
| 11 | 118 | 165 | 1.0 | heel | 5 | 20 | 160 | 1.3 |
| 12 | 120 | 170 | 1.0 | heel | 5 | 18 | 160 | 1.5 |
| 13 | 130 | 175 | 1.0 | heel | 5 | 16 | 160 | 1.7 |
| 14 | 140 | 180 | 1.0 | mid-foot | 5 | 16 | 160 | 1.9 |
| 15 | 150 | 180 | 0.9 | mid-foot | 5 | 16 | 160 | 2.2 |
| 16 | 160 | 182 | 0.9 | mid-foot | 5 | 16 | 160 | 2.4 |
| 17 | 170 | 184 | 0.9 | mid-foot | 5 | 18 | 160 | 2.6 |
| 18 | 180 | 185 | 0.9 | front-foot | 5 | 18 | 160 | 2.9 |
| 19 | 196 | 186 | 0.8 | front-foot | 5 | 20 | 160 | 3.1 |

In this example, left-right balance is the ratio between the ground contact time of the left foot and that of the right foot. In other embodiments, the left-right balance can also, for instance, refer to the step length difference between the right and left foot or to a different parameter related to a balance or imbalance of left and right. The upper body angle is the angle with respect to the vertical and the pelvic rotation is the angle around the vertical axis. EMG frequency is, in this example, the median frequency.

A measured parameter used for the algorithm to determine the fatigue level by fatigue level determination unit 20 is called “P”, and Pᵢ (with i=1 . . . N) is used to refer to each parameter separately. In the example, P₁=sp, P₂=HR, P₃=ca, P₄=lrb, P₅=fl, P₆=uba, P₇=pr, P₈=Ef and P₉=Ea. Let's now define the function f, which gives the fatigue level, as

$$f = {\sum }_{i = 2}^{N} ( \pm 1 \cdot {w}_{i} ( {P}_{i \_ P 1 = c} - {P}_{i \_ r \_ P 1 = c} ) )$$

which is the sum (i=2 . . . N) of plus or minus one times a parameter-dependent weighting factor wᵢ times the difference between Pᵢ and its value at rest for a certain value c of P₁. In a preferred embodiment, P₁ is the speed. Then Pᵢ_{_}_{P1=c} (i=2 . . . N) are the values of the measured parameters at the speed the subject is running at that moment and Pᵢ_{_}ᵣ_{_}_{P1=c} are these values for a situation where he would be rested. +1 (right after the summation sign) is used when a higher value of Pᵢ than Pᵢ_{_}ᵣ hints to a higher fatigue level (examples are heart rate and EMG amplitude), while −1 is used when a lower value of Pᵢ than Pᵢ_{_}ᵣ hints to a higher fatigue level (examples are cadence and EMG median frequency). Parameters that don't have a value should of course be mapped to a value. For example, foot landing on the heel might get value 2, foot landing on the mid-foot might get value 1, and foot landing on the front foot might get value 0. As an example we use the example, where at a certain moment in time the subject is running at a speed of 15 km/h with a heart rate of 170 bpm, a cadence of 175 bpm, a left-right balance of 0.8, heel landing, an upper body angel of 10°, a pelvic rotation of 20°, an EMG median frequency of 140 Hz and an EMG amplitude of 3.1 V, while his normal (rested) values at 15 km/h are 150 bpm, 180 bpm, 0.9, mid-foot, 5°, 16°, 160 Hz, and 2.2 V respectively. Assume furthermore that the weighting factors used by the system are 1.0 bmp⁻¹, 2.0 bpm⁻¹, 15.0, 10.0, 1.0/°, 1.5/°, 0.7 Hz⁻¹ and 5.0 V⁻¹ respectively. This would then give a value for f of:

f=1.0·(170−150)−2.0·(175−180)−15.0·(0.8−0.9)+10.0·(2−1)+1.0·(10−5)+1.5·(20−16)−0.7·(140−160)+5.0·(3.1−2.2)=71.

As this is higher than the threshold of 50 mentioned above as the threshold for ‘high fatigue level allowed’, the subject could then be notified by user notification unit 50 that he should stop, even if a high fatigue level would be allowed during the workout. He would get a warning already when f crosses 50, for instance. If the threshold was set at 10 (very low fatigue level allowed), the warming would already appear for very minor changes in parameters 2 to 9 compared to the normal (rested) values. In that case, if only the heart rate is increased by 10 bpm compared to the normal situation, a warning would appear, even if all other parameters are still normal (i.e. the same as in a rested situation).

The weighting factors wᵢ may be constants for the system or they may be personalized e.g. depending on how experienced the subject is with running and/or even develop slowly in time (i.e. change slowly over periods of weeks or months, for example because the runner is getting used to running). The reason for this is that when a person has never run before and he starts running, his upper body angle (and similarly possibly other parameters) might vary a lot during the training, while this might just be because he is trying to find out what the best way to run is instead of having anything to do with getting tired. Therefore, in that case, the weighting factor of the upper body angle should be low. Next to that, the weighting factors will depend on the type of sport; for cycling the weighting factors will be different than for running. Besides other parameters might be used for different sports and/or for different applications, such as medical applications.

This approach also gives the possibility to still have a working over-exercise indicator even if data are missing (for example due to bad sensor contact with the skin or hampered streaming of the data to the system), as will be explained in the next two sections. Suppose that parameter P₂ is missing. So in the example above, the heart rate would be missing. This is something that could occur for example due to a bad skin contact of a chest strap or due to bad blood perfusion of the skin when an optical heart rate sensor is used. As these data are missing, the term corresponding to that parameter in formula f will be missing. The system could just set this term at zero. As now the formula is made of less (non-zero) terms, the value of the threshold should also be reduced. For example, while originally ‘high fatigue level allowed’ corresponded to a threshold value of 50, this could be adapted to 40 when the heart rate data are missing.

In this case, fatigue level threshold determination unit 30 is adapted to reduce the fatigue level threshold based on the reduced amount of parameters. In another example, fatigue level threshold determination unit 30 can maintain the fatigue level threshold as before and fatigue level determination unit 20 can adapt or scale the fatigue level accordingly, in other words, a fatigue level of 40 determined by fatigue level determination unit 20 could be scaled to correspond the value of 50, which would be the fatigue level in case all parameters would have been considered.

Suppose now that parameter P₁ is missing. In the previous example, the measured parameters will be compared with those parameters in a rested state based on the looked-up parameters for a speed of 15 km/h. In case the speed information is missing, for instance due to a missing GPS signal, another parameter should take over the role of the speed information, based on which the remaining parameters are looked for.

The best choice for P₁ is a parameter that (for the particular subject) changes with speed but is hardly influenced by fatigue (at least for the particular subject). For example, the table indicated above shows that the higher the speed is, the higher the EMG amplitude (Ea) becomes and at a speed of 19 km/h, Ea is almost three times larger than Ea at 10 km/h. Suppose now that relative to this, for this particular subject, the changes in Ea due to fatigue are small. For example, at a speed of 15 km/h Ea is 2.4 V when the person is very tired, instead of 2.2 V for a rested state and, likewise, at other speeds, the differences between Ea when the person is tired compared to a rested state are also relatively small. This would then be a suitable parameter to take the role of P₁. The term of this parameter in f then disappears, so the threshold level should also be reduced, like explained in the previous paragraph.

Whatever is the best parameter to take the role of P₁ can be learnt during the times when there are good speed measurements. During those periods, a table like the one provided above is filled and it is also learnt what the influence of fatigue is on the parameters. In other examples, the table can of course comprise additional and/or alternative parameters. Instead of replacing the role of P₁ (speed) by one of the other parameters Pᵢ with i=2 . . . N when speed becomes unavailable, it is also possible to replace the role of P₁ by another parameter Pₒ. For example, the magnitude of the acceleration of the wrist (integrated over one or several seconds) can be used as a surrogate for speed. Also here, this method will especially work well if this other parameter depends considerably on speed but hardly changes with increasing fatigue level. In this case, the original formula and threshold level can be kept (only now P₁=c is replaced by Pₒ=c₂, with c₂ the value of Pₒ that corresponds to the current value of Pₒ during the run of the subject. The system might have learnt directly the correlations between Pₒ and Pᵢ, with i=2 . . . N, or it might use the relationship between Pₒ and P₁ together with the relationships between P₁ and Pᵢ (i=2 . . . N). Note that many sports watches contain an accelerometer and therefore the magnitude of the acceleration of the wrist (integrated over one or several seconds) might be a good choice for Pₒ, as it can easily be measured and for most subjects it has a clear correlation with speed.

As indicated above, instead of or in addition to determining a maximum allowed fatigue level threshold, a minimum required fatigue level threshold could be set. The subject or another user such as his coach could insert in the system that the subject is supposed to do a hard training. This would correspond to a higher value off than when he or his coach would insert in the system that only a minor amount of fatigue is required, e.g. the first case could correspond to a threshold for f of 25 and the latter to 5. The system could constantly show that the minimum required fatigue level has or has not been reached yet or it could give a warning after a certain amount of time if the fatigue level hasn't been reached yet, to encourage the subject to train harder. Especially when the system knows how long the training will be (for example a goal of 1 hour or a route that takes about 1 hour), it could warn at an appropriate time (e.g. after 45 minutes if the minimum required fatigue level hasn't been reached yet).

In alternative embodiments, instead of a look-up table as in the example provided above, functions could be used to relate the different parameters, such as heart rate, speed, running dynamics parameters, posture, foot landing, EMG signals and so on, which are comprised in the exercise state of the subject provided by exercise state providing unit 10. For example, a linear relationship between speed and heart rate or between any two parameters can be assumed and data produced by subject 7 could be fitted to find user-specific parameters such as slope and offset in the linear relationship.

In other examples, more than one look-up table as the table given above can be provided for determining a fatigue level of the subject, for instance, based on environmental conditions. More precisely, a separate look-up table for running uphill, more preferably even multiple separate look-up tables for different uphill slopes, can be provided. However, also alternative or additional environmental parameters can be considered for constructing additional look-up tables, such as running downhill and the like. As already indicated above, if the subject 7 only sporadically runs in hills or has just started using system 1, fatigue level determination unit 20 can be adapted to compare the subject 7 with up/downhill running data of similar subjects. Similar subjects are subjects with similar running behavior on a flat surface as the subject 7 currently using system 1 and preferably also with comparable weight to the weight of subject 7.

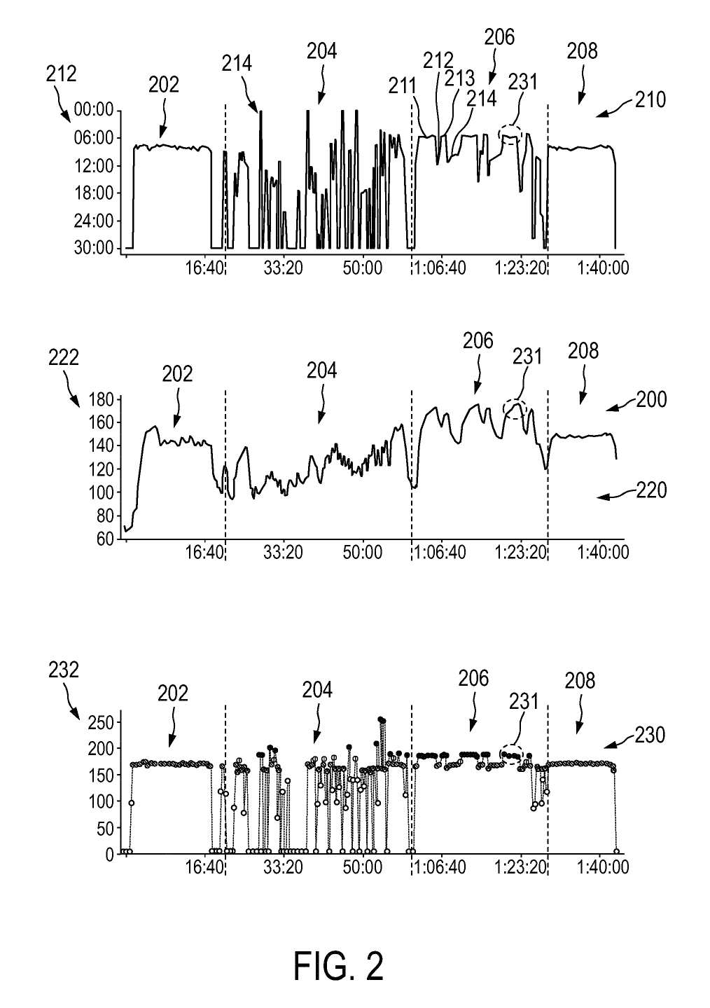

FIG. 2 shows schematically and exemplarily data, out of which overreaching in training, i.e. a high fatigue level of subject 7, can be seen. In this example, FIG. 2 shows an output 200 of a user interface, for instance provided as a web application or the like, based on which a user, for instance subject 7, can further analyze data provided by exercise state providing unit 10, and, for instance, also determine the fatigue level based on the provided exercise state. The exemplary output 200 includes three data representations 210, 220 and 230. Data representation 210 indicates the subject's pace over time, data representation 220 the subject's heart rate over time and data representation 230 the subject's running cadence over time. All three data representations 210, 220 and 230 correspond to the same time interval from 0 to approximately 1 hour and 40 minutes. On the vertical axis of data representation 210 pace 212 is indicated in minutes per mile in this example. A lower value of pace 212 indicates a faster run. A pace approaching 0:0 minutes per mile, as for instance in point 214 is likely to originate from a measurement error, since subject 7 cannot approach these speeds while running.

In data representation 220, the subject's 7 heart rate 222 is indicated, wherein the vertical axis shows heart rates between 60 and 180 beats per minute (bpm).

Finally, data representation 230 indicates the subject's running cadence 232, wherein the vertical axis indicates the subject's running cadence in steps per minute from 0 to 250. In the first 17 minutes, the time region indicated with 202 in the three data representations 210, 220 and 230, subject 7 is doing a warm-up run. From 17 minutes till 60 minutes subject 7 is doing exercises, which is indicated as region 204. Following exercises 204, between 1 hour and 1:30 hours, subject 7 is doing an interval program 206 before finishing the training with a cool-down run during the time region indicated with 208. The interval program 206 consists of three times 1000 meters fast 211, 200 meters slow 212, 300 meters fast 213 and 500 meters slow 214. In the third repetition of the 1000 meter fast interval, which is encircled and labeled 231 in all three data representations 210, 220 and 230, it can be seen that, although having the same speed as for the first and second fast 1000 meters interval 211, the subject's 7 heart rate is higher and the cadence is decreasing more and more. This is a sign of fatigue and the subject should have been notified and stopped the interval program at that time to get most out of the training and to prevent ending up with a considerable recovery time after the training.

In this example, since the subject 7 runs in the same kind of intervals multiple times, data of previous intervals of the same training can be used for comparing and estimating the fatigue level to reach or exceed the fatigue level threshold. In cases, in which the subject 7 does not do repetitions during a training, no good references inside this training are available and historical data, such as from previous trainings, should be used by fatigue level determination unit 20. As also in this example, running trainings frequently contain a part of exercises, in this example the time 204 between 17 minutes and 1 hour. System 1 is adapted to discriminate these exercise part from running by, for instance, accelerometers, since these periods should not be taken into account in the learning process for detecting over-exercise, i.e. for determining the fatigue level. More precisely, during these periods of exercises, subject 7 is varying the motion and motion dynamics, posture, foot landing, et cetera, deliberately.

In other examples, other conditions such as environmental conditions and/or movements which should not be taken into account in the learning process for determining the fatigue level can include a very slippery road due to an occasional very cold day, or running on a very irregular surface with many loose stones, sand, or small rocks, for instance. In these situations, fatigue level determination unit 20 is adapted to exclude these episodes from learning.

System 1 can, in one example, comprise a user input means, for instance a button such as a physical button or a button displayed on a screen of a watch or a phone, or a different input means which can be actuated during the exercising or afterwards to cause the algorithms to exclude the period from the fatigue level determination. Actuating these input means again could lead to fatigue level determination unit 20 to restart data collection and the algorithms again.

In another example, the system 1 detects unusual behavior and user notification unit 50 notifies the user of this unexpected behavior and questions the user whether this unusual behavior is to be taken into account, for instance by means of a voice sound or a respective message on a display. A user or subject 7 can then press “yes” or “no” or can react in other ways, such as by answering with voice.

In another example, unusual episodes can be avoided from being used in the learning process at a later time, for instance while a subject looks at captured running data on his computer, for instance via a web application of which a graphical user interface can produce an output 200 as exemplarily illustrated in and discussed with reference to FIG. 2 above. Subject 7 can then select episodes where he/she was doing exercises or running on a slippery or otherwise irregular surface or under non-ordinary circumstances or environmental conditions to tell the system that these episodes are not to be taken into account for learning for the determination of the fatigue level.

Further preferable, in one example the automatic detection of unusual behavior can be further exploited, such as by asking subject 7 about the origin this unusual behavior is to, wherein subject 7 has options to select in response, wherein the options include for instance “exercises”, “slippery/irregular road”, “strong head wind”, “strong tail wind”, “running uphill”, “running downhill”, “other discard” or “other take into account”. Thereby, a lack of sensors for environmental conditions can be compensated for. Even further, these options can be classified in more detail, or other parameters or options can be provided in other examples. Also this questioning can occur during the subject's 7 exercising, i.e. while the subject 7 is running, or at a later stage, while subject 7 or a different user analyzes his/her data which has been recorded before, by means of, for instance, the computer application shown exemplarily in FIG. 2.

FIG. 3 shows schematically and exemplarily a flow diagram of a method 300 for assisting exercising of a subject. The method comprises providing 310 an exercise state of the subject at a time of determination, determining 320 a fatigue level of the subject based on the exercise state of the subject, determining 330 a fatigue level threshold for the subject at the time of determination, and evaluating 340 the fatigue level in comparison to the fatigue level threshold. Further, method 300 optionally includes notifying 350 the subject of the evaluation of the fatigue level.

In one example, in case the evaluation unit is adapted to determine whether the fatigue level exceeds the fatigue level threshold, the system 1 for assisting exercising of a subject 7 can be referred to as an over-exercise indicator. Preferably, the over-exercise indicator according to the invention uses input on whether the subject is doing a race or a training and, further preferably, also when the next exercise training is planned and/or planned periodization periods of the subject. Further preferably, the intensity level of exercise during the last couple of days can be taken into account.

In the following, two examples will be given to further clarify the benefits of the invention. For the sake of example and not limitation, it will be used that the fatigue level threshold can vary between 0 and 50, where 50 is allowance of a very high fatigue level, while 10 is allowance of only a low fatigue level,

### Example 1

Suppose that a user has been exercising very hard for a couple of weeks. It is time to give his body some rest to recover well from the exercise in the past weeks. Therefore the fatigue level threshold is set to 10 for the exercise sessions that he will do in this week, while it was fluctuating between 40 and 50 for the exercise sessions in the past weeks.

### Example 2

Suppose that a user was planning to exercise hard in the coming days. His fatigue level threshold was set to 40 for a training session on Monday, 50 for a training session on Tuesday, and 45 for a training session on Wednesday. However, after the training session of Tuesday, the user becomes ill. He still doesn't feel well on Wednesday. He could let the system know that he doesn't feel well by giving user input to the app belonging to the system, or the system could even measure that his state isn't good, for example by measurement of an elevated resting heart rate. In response to this user input or measurement, the system lowers down the fatigue level threshold for Wednesday to 10, because the user should not do a hard training on Wednesday, as then he will not recover from his illness.

The fatigue level thresholds determined by the fatigue level threshold determination unit can be based both on thresholds that were set already in the week(s) before the exercise session as well as on adjustments to these thresholds for instance on the same day of or even in real-time with the exercise session.

Preferably, the over-exercise indicator then can learn the subject's fitness level and running style.

Further preferably, all needed parameters for determining the exercise state of the subject can be measured with in-ear sensors and audio feedback is given via the ear as well.

Preferably, the over-exercise indicator takes into account influences from environmental factors such as wind, altitude, temperature and inclination for determining a fatigue level and/or a fatigue level threshold for the subject.

Preferably, the over-exercise indicator uses a change in output of skeletal muscles additionally or alternatively to classical parameters, namely speed, duration, heart rate and/or HRV. Preferably, the output of skeletal muscles can be one or more of running dynamics, foot landing, posture, muscle tension, muscle fatigue or other measures that can be derived from features in an EMG signal.

Although the preceding paragraphs have been particularly described as if they were tailored to running, the over-exercise indicator and the more general system 1 for assisting exercising of a subject 7, the application is not limited to running and similarly holds for other sports, such as swimming or the like. Further, it has been described with the particular example of the subject carrying the system 1 along, in other examples, also applications in which the user is different from the subject. For instance, system 1 can be applied to a soccer game to determine whether the soccer player, being the subject in this case, should have a rest. In this example, for instance, the soccer coach, being a user, could be provided with the evaluation result to determine whether players on the field during the match are to be exchanged. The range of possible applications is of course not limited and the above are just a very narrow range of contemplated applications.

Although generally accelerometers have been described as means for determining a motion signal of the subject, also different kinds of motion sensors are contemplated. For example, instead of or alternatively to an accelerometer, also other kinds of inertia sensors, such as sensors comprising a gyroscope, can be employed in embodiments of the invention.

In summary, one key advantage of the present invention is that the threshold for giving a warning for fatigue is variable; more precisely: the fatigue level threshold is variable. For example, when the user is going to participate in a race the next day, he shouldn't get tired today and therefore the system will warn him to stop already at a very low level of fatigue. On the other hand, when the next important race will take place one month from now and the user is therefore in a heavy training period now, the system only warns to stop the training when the user is already at a very high fatigue level. Therefore, the invention comprises a fatigue level threshold determination unit. This fatigue level threshold determination unit determines which should be the threshold for, for instance, a warning, based preferably on at least one of an exercise history, a planned activity (think of periodization or tapering), and a parameter of the subject.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Exercise state providing unit 10, fatigue level determination unit 20, fatigue level threshold determination unit 30 and evaluation unit 40 can, in one example, be implemented on a sports watch and/or a sports tracking application which can be installed on a mobile phone, for instance. However, in other examples, one, more or all of the exercise state providing unit 10, fatigue level determination unit 20, fatigue level threshold determination unit 30 and evaluation unit 40 can be implemented on a server and accessed, for instance, via a web interface using a mobile phone, or a portable or stationary computer device. In this example, data provided by exercise state providing unit 10 can be stored on a database on the server. Preferably, system 1 allows for a combination of both such that exercising of subject 7 can be evaluated in real-time using an exercise state providing unit 10, a fatigue level determination unit 20, a fatigue level threshold determination unit 30 and an evaluation unit 40 which are accessible while exercising, and system 1 can be employed to evaluate the exercising at a later stage by subject 7 or a different user, for example by accessing system 1 for instance via a web application.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. A system for assisting exercising of a subject, wherein the system comprises:
an exercise state providing unit for providing an exercise state of the subject for or during an exercise session,
a fatigue level determination unit for determining a fatigue level of the subject based on the exercise state of the subject,
a fatigue level threshold determination unit for determining a fatigue level threshold for the subject for the exercise session, and
an evaluation unit for evaluating the fatigue level in comparison to the fatigue level threshold.

2. The system according to claim 1, wherein the fatigue level threshold determination unit is adapted to determine the fatigue level threshold based on at least one of an exercise history of the subject, a planned activity and a parameter of the subject.

3. The system according to claim 2, wherein the determination based on the planned activity includes at least one of periodization and tapering prior to a future activity.

4. The system according to claim 1, wherein the fatigue level threshold determination unit is adapted to determine a maximum fatigue level threshold and/or a minimum fatigue level threshold.

5. The system according to claim 1, wherein the exercise state providing unit is adapted to provide the exercise state of the subject based on at least two parameters, wherein the at least two parameters correspond to at least two different of the following groups:
speed, heart rate and heart rate variability,
running dynamics,
foot landing,
posture, and
electromyography related parameters.

6. The system according to claim 5, wherein parameters corresponding to the group of running dynamics comprise a ground contact time, a vertical oscillation, a cadence, a stride length, a stride interval, a stride interval variability, a left-right balance, a measure for braking, a step length, a step interval and a step interval variability.

7. The system according to claim 5, wherein parameters corresponding to the group of posture comprise an angle of upper body, a pelvic rotation and a head orientation.

8. The system according to claim 1, wherein the exercise state providing unit is adapted to provide the exercise state of the subject based on at least one of a foot landing parameter and a head orientation parameter, wherein the exercise state providing unit is configured to determine the at least one of a foot landing parameter and a head orientation parameter based on an inertia signal.

9. The system according to claim 1, wherein the exercise state providing unit comprises an exercise state measurement unit, wherein the exercise state measurement unit is adapted to be mounted in-ear of the subject.

10. The system according to claim 1, wherein the fatigue level determination unit is adapted to provide the fatigue level of the subject based on past exercise states of the subject.

11. The system according to claim 1, wherein the fatigue level determination unit is adapted to provide a reference exercise state of the subject and to provide the fatigue level based on a deviation of the exercise state from the reference exercise state.

12. The system according to claim 1, wherein the exercise state and the reference exercise state of the subject each comprise a set of at least two parameters, wherein the fatigue level determination unit 2 is adapted to provide the fatigue level based on a weighted sum of differences between each set of corresponding parameters among the reference exercise state parameter set and the exercise state parameter set.

13. The system according to claim 1, wherein the fatigue level determination unit is adapted to determine the fatigue level based on environmental influences and/or the fatigue level threshold determination unit is adapted to determine the fatigue level threshold based on environmental influences.

14. A method for assisting exercising of a subject, wherein the method comprises:
providing, by an exercise state providing unit, an exercise state of the subject for or during an exercise session,
determining, by a fatigue level determination unit, a fatigue level of the subject based on the exercise state of the subject,
determining, by a fatigue level threshold determination unit, a fatigue level threshold for the subject for the exercise session, and
evaluating, by an evaluation unit, the fatigue level in comparison to the fatigue level threshold.

15. A computer program for assisting exercising of a subject, the computer program comprising program code means for causing a system as defined in claim 1, when the computer program is run on the system.
